SEQUENCE LISTING (6 sequences)

SEQ ID NO: 1 - DNA, 744 bp, Homo sapiens
gagtatttgaggctcggagccaccgccccgccggcgcccgcagcacctcctcgccagcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccctggagcacctggagcttatcccggagcacctgcacctggagtctacccagggccacccagcggccctggggcctacccatcttctggacagccaagtgccaccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcacttacatgtgtaaaggtttcatgttcactgtgagtgaaaatttttacattcatcaatatccctcttgtaagtcatctacttaataaatattacagtgaattacctgtctcaatatgtcaaaaaaaaaaaaaaaaaa

SEQ ID NO: 2 - protein, 250 aa, Homo sapiens [sequence omitted]

SEQ ID NO: 3 - DNA, 814 bp, Homo sapiens
agcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctcataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaa

SEQ ID NO: 4 - protein, 250 aa, Homo sapiens
MADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 5 - DNA, 29 bp, Artificial Sequence
  Synthetic primer
ggatccgcagtgattgtgcctaataacct

SEQ ID NO: 6 - DNA, 31 bp, Artificial Sequence
  Synthetic primer
aagctttatcatggtatatgaagcactggtg

(19) United States
(12) Patent Application Publication
(10) Pub. No.: US 2016/0122407 A1
(43) Pub. Date: May 5, 2016

(54) Galectin-3 Inhibitor (Gal-3M) is Associated with Additive Anti-Myeloma and Anti-Solid Tumor Effects, Decreased Osteoclastogenesis and Organ Protection when Used in Combination with Proteasome Inhibitors

(71) Applicant: Texas Tech University System, Lubbock, TX (US)

(72) Inventors: Maurizio Chiriva-Internati, Lubbock, TX (US); Jose A. Figueroa, Lubbock, TX (US); Everardo Cobos, Lubbock, TX (US)

(21) Appl. No.: 14/992,173

(22) Filed: Jan. 11, 2016

Related U.S. Application Data
(63) Continuation of application No. 14/612,757, filed on Feb. 3, 2015.
(60) Provisional application No. 61/934,927, filed on Feb. 3, 2014.

(51) Int. Cl. C07K 14/47

(52) U.S. Cl. CPC C07K 14/4726

(57) ABSTRACT

The present invention includes a method for the treatment of myeloma comprising identifying a patient with myeloma and administering to the patient a synergistic, effective amount of a truncated, dominant negative form of Galectin-3 and a proteasome inhibitor.

### CROSS-REFERENCE TO RELATED APPLICATIONS

This application claims priority based on U.S. Provisional Application No. 61/934,927, filed Feb. 3, 2014, and U.S. patent application Ser. No. 14/612,757, filed Feb. 3, 2015, the entire contents of each of which are incorporated by reference in their entirety.

### TECHNICAL FIELD OF THE INVENTION

The present invention relates in general to the field of treatments for myeloma, and more particularly, to the use of Galectin-3M in conjunction with proteasome inhibitors in myeloma and solid tumor therapy.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Feb. 3, 2015, is named TECH1112CON1_SeqList and is 8 kilobytes in size.

### BACKGROUND OF THE INVENTION

Without limiting the scope of the invention, its background is described in connection with myeloma.

Galectins are S-type lectins that bind β-galactose-containing glycoconjugates. Since the discovery of the first galectin in animal cells in 1975, fifteen mammalian galectins have been isolated. They regulate different biological processes such as cell adhesion, regulation of growth, apoptosis, tumor development and progression. Accumulating evidences report multiple roles for Galectin in cancer. Among them, Galectins-3 is an attractive target, since it is involved in many features of tumor progression such as adhesion, proliferation, and metastasis.

U.S. Pat. No. 6,770,622, issued to Jarvis, et al., is directed to an N-terminally truncated Galectin-3 for use in treating cancer. Briefly, this patent teaches a composition having an effective amount of N-terminally truncated Galectin-3 in a pharmaceutically acceptable carrier. Also provided by the present invention is a method of treating cancer by administering to a patient in need of such treatment an effective amount of N-terminally truncated Galectin-3 in a pharmaceutically acceptable carrier. Data is provided that shows the treatment of breast cancer cells in a mouse model system.

International Patent Publication No. WO2012135528 A2, by the present inventors, is directed to the use of Galectin-3C in combination therapy for human cancer, in which Galectin-3C was used in combination with a proteosome inhibitor, the combination having a pharmacologic activity greater than the expected additive effect of its individual components. Other embodiments of the invention provide novel synergistic compositions of Galectin3C with a proteasome inhibitor capable of reducing or overcoming resistance that develops to the proteasome inhibitor or reducing the adverse side effects from the proteasome inhibitor through increasing the therapeutic efficacy of lower doses.

Others have published results that show that Galectin-3C was used to treat multiple myeloma, including the use of Galectin-3C in conjunction with the chemotherapeutic Bortezomib, Mirandola, L., et al. “Galectin3C Inhibits Tumor Growth and Increases the Anticancer Activity of Bortezomib in a Murine Model of Human Multiple Myeloma” Plos One, July 2011|Volume 6|Issue 7|e21811.

Multiple myeloma (MM) is a neoplastic plasma cell disorder which results in end-organ damage, including renal insufficiency and skeletal destruction. The treatment of MM is complex and exploits different approaches, including the use of standard cytotoxic agents, autologous and allogeneic bone marrow stem cell transplantation, immune-modulating agents and proteasome inhibitors. Carfilzomib, a new proteasome inhibitor with significant anti-myeloma and anti-osteoclastogenic properties, has recently been approved for treatment of MM patients. Unfortunately, carfilzomib has also been associated with significant toxicities, including renal and cardiac insufficiency.

### SUMMARY OF THE INVENTION

The present inventors have shows previously that Galectin-3 inhibition results in significant anti-myeloma effects in a xenograft in vivo model. The present invention uses a new Galectin-3 inhibitor, Gal3M, to not only interfere with MM cell growth and migration in vitro, but is also associated with MM cell growth retardation in an in vivo syngeneic model of this disease. It was found that the combination of Gal3M and carfilzomib not only had additive anti-myeloma effects, but was also associated with cardio-renal protection against carfilzomib-induced damage. Moreover, the use of this combination resulted in net osteoclastic inhibition and increased bone formation in an in vivo model. Thus, multiple beneficial effects were found when using this novel combination in the treatment of MM and other solid tumors.

In one embodiment, the present invention includes a method for the treatment of a cancer comprising: identifying a patient with cancer; and administering to the patient an effective amount of truncated, dominant negative form of Galectin-3 and a proteasome inhibitor. In one aspect, the truncated, dominant negative form of Galectin-3 is administered by intravenous or intraperitoneal route. In another aspect, the cancer is a myeloma that is drug resistant. In another aspect, the cancer is a myeloma that is multiple-drug resistant. In another aspect, the cancer is a myeloma that is an advanced, refractory myeloma cancer. In another aspect, the amount of the truncated, dominant negative form of Galectin-3 is sufficient to reduce cardiac or renal toxicity. In another aspect, the truncated, dominant negative form of Galectin-3 is provided as a nucleic acid vector having SEQ ID NO.: 3, and expressed as SEQ ID NO.: 4. In another aspect, the truncated, dominant negative form of Galectin-3 is provided as a polypeptide having SEQ ID NO.: 4. In another aspect, the truncated, dominant negative form of Galectin-3 is provided as a nucleic acid in an expression vector that expresses the truncated, dominant negative form of Galectin-3 upon entry into a cell. In another aspect, the combination of truncated, dominant negative form of Galectin-3 and the a proteasome inhibitor is synergistic. In another aspect, the combination of truncated, dominant negative form of Galectin-3 and a proteasome inhibitor triggers at least one of osteoclastic inhibition or increased bone formation. In another aspect, the proteasome inhibitor is selected from bortezomib, carfilzomib, disulfiram, epigallocatechin-3-gallate, salinosporamide A, epoxomicin, lactacystin, MG132, ONX 0912, CEP-18770, and MLN9708. In another aspect, the proteasome inhibitor is carfilzomib. In another aspect, the cancer is a hematological cancer, solid tumor, or a sarcoma. In another aspect, the cancer is selected from: breast cancer colon cancer, colorectal carcinomas, non-small cell lung cancer, small-cell lung cancer, liver cancer, ovarian cancer, prostate cancer, uterine cervix cancer, urinary bladder cancer, gall bladder carcinoma, gastric carcinoma, esophageal cancer, gastrointestinal stromal tumor, pancreatic cancer, germ cell tumors, mast cell tumors, neuroblastoma, retinoblastoma, mesothelioma, mastocytosis, testicular cancers, glioblastomas, astrocytomas, sarcoma, osteosarcoma, B cell lymphoma, T cell lymphoma, NK cell lymphoma, Hodgkin's lymphoma, non-Hodgkin's lymphoma, melanoma, basal cell carcinoma, skin cancer, myeloma, leukemia, acute myelocytic leukemia (AML), acute lymphocytic leukemia (ALL), myelodysplastic syndrome, chronic lymphocytic leukemia (CLL) and chronic myelogenous leukemia (CML). In another aspect, the Galectin-3 has the following mutations P113, L114 and Y118.

Another embodiment of the present invention includes a method for the treatment of cancer, comprising: identifying a patient with cancer; and administering to the patient an effective amount of truncated, dominant negative form of Galectin-3 and a proteasome inhibitor, without a proteasome inhibitor-associated cardiac or renal toxicity. In one aspect, the truncated, dominant negative form of Galectin-3 is administered by intravenous or intraperitoneal route. In another aspect, the cancer is a myeloma that is drug resistant. In another aspect, the cancer is a myeloma that is multiple-drug resistant. In another aspect, the truncated, dominant negative form of Galectin-3 is provided as a nucleic acid vector having SEQ ID NO.: 3, and expressed as SEQ ID NO.: 4. In another aspect, the truncated, dominant negative form of Galectin-3 is provided as a polypeptide having SEQ ID NO.: 4. In another aspect, the truncated, dominant negative form of Galectin-3 is provided as a nucleic acid in an expression vector that expresses the truncated, dominant negative form of Galectin-3 upon entry into a cell. In another aspect, the combination of truncated, dominant negative form of Galectin-3 and Carfilzomib is synergistic. In another aspect, the combination of truncated, dominant negative form of Galectin-3 and Carfilzomib triggers at least one of osteoclastic inhibition or increased bone formation. In another aspect, the proteasome inhibitor is selected from bortezomib, carfilzomib, disulfiram, epigallocatechin-3-gallate, salinosporamide A, epoxomicin, lactacystin, MG132, ONX 0912, CEP-18770, and MLN9708. In another aspect, the proteasome inhibitor is carfilzomib. In another aspect, the cancer is a hematological cancer, solid tumor, or a sarcoma. In another aspect, the Galectin-3 has the following mutations P113, L114 and Y118. In another aspect, the cancer is selected from: breast cancer, colon cancer, colorectal carcinomas, non-small cell long cancer, small-cell lung cancer, liver cancer, ovarian cancer, prostate cancer, uterine cervix cancer, urinary bladder cancer, gall bladder carcinoma, gastric carcinoma, esophageal cancer, gastrointestinal stromal tumor, pancreatic cancer, germ cell tumors, mast cell tumors, neuroblastoma, retinoblastoma, mesothelioma, mastocytosis, testicular cancers, glioblastomas, astrocytomas, sarcoma, osteosarcoma, B cell lymphoma, T cell lymphoma, NK cell lymphoma, Hodgkin's lymphoma, non-Hodgkin's lymphoma, melanoma, basal cell carcinoma, skin cancer, myeloma, leukemia, acute myelocytic leukemia (AML), acute lymphocytic leukemia (ALL), myelodysplastic syndrome, chronic lymphocytic leukemia (CLL) and chronic myelogenous leukemia (CML).

Yet another embodiment of the present invention includes a method of performing a clinical trial to evaluate a candidate drug believed to be useful in treating myeloma, the method comprising: (a) measuring from tissue suspected of having a myeloma from a set of patients; (b) administering a candidate drug to a first subset of the patients, and a placebo to a second subset of the patients, wherein the candidate substance is at least one of a truncated or a dominant negative form of Galectin-3 and a proteasome inhibitor, without a proteasome inhibitor-associated cardiac or renal toxicity; (c) repeating step a) after the administration of the candidate drug or the placebo; and (d) determining if the candidate drug reduces at least one of the number or proliferation of myeloma cells that is statistically significant as compared to any reduction occurring in the second subset of patients, wherein a statistically significant reduction indicates that the candidate drug is useful in treating the myeloma without a proteasome inhibitor-associated cardiac or renal toxicity.

In one embodiment, the present invention includes a protein comprising a Galectin-3 inhibitor having a sequence that is 95% identical to Galectin-3 with the following mutations P113, L114 and Y118. In one aspect, the protein has 90%, 97%, 98%, 99%, or 100% sequence identity to Galectin-3 with the following mutations P113, L114 and Y118.

In one embodiment, the present invention includes a nucleic acid encoding a Galectin-3 inhibitor having a sequence that is 95% identical to Galectin-3 with the following mutations P113, L114 and Y118. In one aspect, the nucleic acid has 96%, 97%, 98%, 99%, or 100% sequence identity to Galectin-3 with the following mutations P113, L114 and Y118.

In one embodiment, the present invention includes a host cell comprising a Galectin-3 inhibitor having a sequence that is 95% identical to Galectin-3 with the following mutations P113, L114 and Y118. In one aspect, the host cell expresses the Galectin-3 inhibitor having a sequence that is 95% identical to Galectin-3 with the following mutations P113, L114 and Y118.

In one embodiment, the present invention includes a method of making a protein comprising expressing in a host cell a Galectin-3 inhibitor having a sequence that is 95% identical to Galectin-3 with the following mutations P113, L114 and Y118.

### BRIEF DESCRIPTION OF THE DRAWINGS

For a more complete understanding of the features and advantages of the present invention, reference is now made to the detailed description of the invention along with the accompanying figures and in which:

FIGS. 1A to 1C show the characterization of 5TGM1 cells. 5TGM1 cells shown high expression of Galectin-3 both at RNA (FIG. 1A, RT-PCR) and protein (FIG. 1B, Western Blot) level The CTA SP17 expression was tested as a marker for MM cells. Results were farther confirmed by immunofluorescence (FIG. 1C). Figures are representative of at least three experiments with similar results.

FIGS. 2A and 2B show the combination of Carfilzomib and Galectin3M enhancing the effects of these two drugs on 5TGM1 cells. Cells were treated with Carf (10 nM), Gal3M (10 ug/ml) and with Carf+Gal3M. Results show that growth (a), chemotaxis, (b) and invasion (c) ability of 5TGM1 were significantly inhibited in presence of Carf and Gal3M. Moreover, the combination of these two drugs has a synergic effect on all those cancer-related process. Experiments were done in triplicate and repeated three times. Error bars represent ±SD. Statistical analysis was performed with One-way anova and Turkey post-test (***=p<0.001).

FIGS. 3A and 3B show that Carfilzomib and Gal3M combination enhances the effects of those drugs on tumor growth in vivo. FIG. 3A shows the effects of the treatments on the IgG2 serum levels, as index of tumor cells engraftment and growth. SP17 levels in the serum were used as marker of tumor growth (FIG. 3B). Statistical analysis was performed with two-failed t-Test and Two-way Anova and Bonferroni post-test (*=p<0.5; **=p<0.01; ***=p<0.001).

FIGS. 4A and 4B show the effects of Carfilzomib, Gal3M and Carf+Gal3M on kidney toxicity. FIG. 4A shows the Eosin/Hematoxylin staining was performed on section of kidneys from 5TGM1 mice for the morphological evaluation. All samples were also stained for active Caspase3, as index of apoptosis (FIG. 4B). Representative pictures were taken at 40× magnification. Carfilzomib has shown to have kidney toxicity both at minimum and maximum dose, whereas Gal3M is not only non-toxic, but has also a protective effect on both organs from the side effects due to the treatment with Carfilzomib.

FIGS. 5A and 5B show the effects of Carfilzomib, Gal3M and Carf+Gal3M on heart toxicity. FIG. 5A shows the Eosin/Hematoxylin staining was performed on section of kidneys from 5TGM1 mice for the morphological evaluation. All samples were also stained for active Caspase3, as index of apoptosis (FIG. 5B). Representative pictures were taken at 40× magnification. Carfilzomib has shown to have kidney toxicity both at minimum and maximum dose, whereas Gal3M is not only non-toxic, but has also a protective effect on both organs from the side effects due to the treatment with Carfilzomib.

FIG. 6 shows the effects of Carf, Gal3M and their combination on 5TGM1-driven osteoclastogenesis in vitro. Raw264.7 cells were co-cultured with 5TGM1 cells and treated for 5 days (FIG. 6 top panels). TRAP staining and count of multinucleated cells (FIG. 6 bottom panels). The combination of Carf and Gal3M is able to revert the side effects of Gal3M on osteoclasts development. Error bars represent standard deviations calculated out of 3 independent experiments. Statistical analysis was performed with One-way anova and Turkey post-test (*=p<0.5).

FIG. 7 shows the effects of the different treatments on MM-associated skeletal destruction. The top left panel of FIG. 7 shows the bone density BMD was calculated from X-Ray images. ROI1 represents foe trabecular area near the proximal femur end. ROI2 represents the trabecular area near the distal femur end. ROI3 represents the trabecular area near the proximal tibia end. Carfilzomib is able to revert the side effects of Galectin3M on bone resorption, indeed the combination of Carf and Galectin3M causes a reduction in skeletal resorption in 5TGM1 mice. Statistical analysis was performed with Two-way ANOVA and Bonferroni post-test (*=p<0.5; **=p<0.01;***=p<0.001). The bottom panels show the results were further validated by IHC analysis on decalcified bones for Osteoclacin (B) and Cathepsin K (C). Figures are representative of at least 5 sections.

FIG. 8 is a flow chart for a study of murine lung cancer with Gal3M and Carfilzomib.

FIG. 9 is a graph that shows the change in tumor volume with no treatment and with 112 or 224 ug of Carfilzomib.

FIG. 10 is a graph that shows the change in tumor volume with no treatment and with 112 or 224 ug of Carfilzomib and XM.

FIG. 11 is a graph that shows the treatment of a solid tumor with no treatment, XM alone, Carfilzomib alone (112 or 224 ug), and Carfilzomib 112 or 224 ug plus XM.

FIG. 12 is a graph that shows the change in tumor volume with no treatment and with treatment with XM.

FIG. 13 is a graph that compares the toxicity of no treatment, XM alone, Carfilzomib alone, and Carfilzomib plus XM.

FIG. 14 is a Western Blot of the apoptopic marker caspacse-3 of cells in solid tumors with no treatment, XM alone, Carfilzomib alone, and Carfilzomib plus XM.

FIG. 15 shows two graphs, in the left panel the percentage survival of with no treatment, XM alone, Carfilzomib alone, and Carfilzomib plus XM (left panel), and on the right panel the toxicity of the no treatment, XM alone, Carfilzomib alone, and Carfilzomib plus XM.

FIG. 16 is a reverse transcriptase-PCR for Sp17 in 5gtm mice, lane 1 5gtm1 cells, lane 2 no RT, lane 3 no template, lane 5 bone and lane 5 spleen.

FIG. 17 is a graph that shows the results of an ELISA for AKAP-4 expression in tumor and lung tissue.

FIG. 18 shows five graphs that show the expression of the listed cell lines as a function of the amount of XM provided.

FIG. 19 shows a dose response curve of % ATP content for the various cell lines as a function of XM provided.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The terms “administration of” or “administering a” compound should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms: inhalation powders, sprays, suspensions, and the like; and rectal suppositories.

The terms “effective amount” or “therapeutically effective amount” means the amount of the subject compound mat will elicit the biological or medical response of a tissue, system, animal, or human that is being sought by the researcher, veterinarian, medical doctor or other clinician. As used herein, the term “treatment” refers to the treatment of the mentioned conditions, particularly in g patient who demonstrates symptoms of the disease or disorder.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology anchor symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

As used herein, a “pharmaceutically acceptable” component is one that is suitable for use with humans and/or animals without undue adverse side effects (such as toxicity, irritation, and allergic response) commensurate with a reasonable benefit/risk ratio.

Multiple myeloma is a malignant plasma-cell disorder characterized by clonal proliferation of neoplastic plasma cells (PCs) in the bone marrow (BM). MM accounts for 1% of all cancers and about 10% of all hematologic malignancies (Rajkumar, 2012). Despite recent advances in its treatment, myeloma remains incurable, with a median survival of 5 years after diagnosis (Zeng, 2013).

Osteoclastogenesis and osteoblastogenesis are finely orchestrated processes that are clearly disrupted in MM, with increased activation of osteoclasts and suppression of bone formation in areas adjacent to tumor foci being hallmarks of the disease. These interactions between MM cells and BM microenvironment establish a “vicious cycle” resulting in skeletal destruction, MM cell growth and the development of drug resistance (Colombo and Mirandola, 2013).

Carfilzomib (Kyprolis™, Onyx Pharmaceuticals, South San Francisco, Calif.) is a new selective and irreversible proteasome inhibitor recently approved by the US Food and Drug Administration (FDA) as single-agent treatment for relapsed or refractory multiple myeloma (RRMM). Carfilzomib has been associated with potentially significant renal and cardiac side effects leading to discontinuation of therapy in approximately 15% of the treated patients (Redic, 2013). Indeed, cardiac failure has been reported in carfilzomib-treated patients, some of these events resulting in fatal outcome. Since approximately 60% of MM patients involved in carfilzomib clinical trials showed some degree of renal dysfunction at the time of enrollment, the need for careful patient selection and strategies that may result in decreased toxicity and organ protection when using this important drug is evident (Redic, 2013).

The inventors have previously demonstrated the ability of Galectin-3C, a dominant negative inhibitor of Galectin-3, to inhibit MM growth in vitro and in vivo (Mirandola, 2011). Galectin-3C alone and in combination with bortezomib resulted in more than 90% growth inhibition of MM growth in a xenograft mouse model of human MM. Importantly, Galectin-3 appears to have anti-osteoclastogenic effects (Li Y J, 2009) and is a mediator of both cardiac dysfunction (Ahmad, 2012) and renal fibrosis (Kolatsi-Joannou, 2011) in humans. The present inventors combined carfilzomib and Galectin-3 inhibitors in MM to evaluate then combined anti-myeloma effects.

The present invention shows the effects of Galectin-3M (Gal3M), a new and more efficient dominant inhibitor of Galectin-3, alone and in combination with carfilzomib, in the murine syngeneic model of MM, 5TGM1.

Galectin-3 is encoded by a single gene, LGALS3, located on chromosome 14, locus q21-q22, having cDNA sequence:

(SEQ ID NO. 1)
GAGTATTTGA GGCTCGGAGC CACCGCCCCG CCGGCGCCCG
CAGCACCTCC TCGCCAGCAG CCGTCCGGAG CCAGCCAACG
AGCGGAAAAT GGCAGACAAT TTTTCGCTCC ATGATGCGTT
ATCTGGGTCT GGAAACCCAA ACCCTCAAGG ATGGCCTGGC
GCATGGGGGA ACCAGCCTGC TGGGGCAGGG GGCTACCCAG
GGGCTTCCTA TCCTGGGGCC TACCCCGGGC AGGCACCCCC
AGGGGCTTAT CCTGGACAGG CACCTCCAGG CGCCTACCCT
GGAGCACCTG GAGCTTATCC CGGAGCACCT GCACCTGGAG
TCTACCCAGG GCCACCCAGC GGCCCTGGGG CCTACCCATC
TTCTGGACAG CCAAGTGCCA CCGGAGCCTA CCCTGCCACT
GGCCCCTATG GCGCCCCTGC TGGGCCACTG ATTGTGCCTT
ATAACCTGCC TTTGCCTGGG GGAGTGGTGC CTCGCATGCT
GATAACAATT CTGGGCACGG TGAAGCCCAA TGCAAACAGA
ATTGCTTTAG ATTTCCAAAG AGGGAATGAT GTTGCCTTCC
ACTTTAACCC ACGCTTCAAT GAGAACAACA GGAGAGTCAT
TGTTTGCACT TACATGTGTA AAGGTTTCAT GTTCACTGTG
AGTGAAAATT TTTACATTCA TCAATATCCC TCTTGTAAGT
CATCTACTTA ATAAATATTA CAGTGAATTA CCTGTCTCAA
TATGTCAAAA AAAAAAAAAA AAAA

Galectin-3 has an amino acid sequence of:

(SEQ ID NO. 2)
MADNFSLHDA LSGSGNPNPQ GWPGAWGNQP AGAGGYPGAS
YPGAYPGQAP PGAYPGQAPP GAYPGAPGAY PGAPAPGVYP
GPPSGPGAYP SSGQPSATGA YPATGPYGAP AGPLIVPYNL
PLPGGWWPRM LITILGTVKP NANRIALDFQ RGNDVAFHPN
PRFNENNRRV TVCNTKLDNN WGREERQSVF PFESGKPFKI
QVLVEPDHFK VAVNDAHLLQ YNHRVKKLNE ISKLGISGDI
DLTSASYTMI.

Galectin3C has cDNA sequence:

(SEQ ID NO. 3)
AGCAGCCGTC CGGAGCCAGC CAACGAGCGG AAAATGGCAG
ACAATTTTTC GCTCCATGAT GCGTTATCTG GGTCTGGAAA
CCCAAACCCT CAAGGATGGC CTGGCGCATG GGGGAACCAG
CCTGCTGGGG CAGGGGGCTA CCCAGGGGCT TCCTATCCTG
GGGCCTACCC CGGGCAGGCA CCCCCAGGGG CTTATCCTGG
ACAGGCACCT CCAGGCGCCT ACCATGGAGC ACCTGGAGCT
TATCCCGGAG CACCTGCACC TGGAGTCTAC CCAGGGCCAC
CCAGCGGCCC TGGGGCCTAC CCATCTTCTG GACAGCCAAG
TGCCCCCGGA GCCTACCCTG CCACTGGCCC CTATGGCGCC
CCTGCTGGGC CACTGATTGT GCCTTATAAC CTGCCTTTGC
CTGGGGGAGT GGTGCCTCGC ATGCTCATAA CAATTCTCGG
CACGGTGAAG CCCAATGCAA ACAGAATTGC TTTAGATTTC
CAAAGAGGGA ATGATGTTGC CTTCCACTTT AACCCACGCT
TCAATGAGAA CAACAGGAGA GTCATTGTTT GCAATAGAAA
GCTGGATAAT AACTGGGGAA GGGAAGAAAG ACAGTCGGTT
TTCCCATTTG ALAGTGGGAA ACCATTCAAA ATACAAGTAC
TGGTTGAACC TGACCACTTC AAGGTTGCAG TGAATGATGC
TCACTTGTTG CAGTACAATC ATCGGGTTAA AAAACTCAAT
GAAATCAGCA AACTGGGAAT TTCTGGTGAC ATAGACCTCA
CCAGTGCTTC ATATACCATG ATATAATCTG

Galectin-3C has amino acid sequence:

(SEQ ID NO. 4)
MADNFSLHDALSGSGNPNPQGWPGAWGNQPAGAGGYPGASYPGAYPGQAP
PGAYPGQAPPGAYPGAPGAYPGAPAPGVYPGPPSGPGAYPSSGQPSATGA
YPATGPYGAPAGPLIVPYNLPLPGGWWPRMLITILGTVKPNANRIALDFQ
RGNDVAFHPNPRFNENNRRVTVCNTKLDNNWGREERQSVFPFESGKPFKI
QVLVEPDHFKVAVNDAHLLQYNHRVKKLNEISKLGISGDIDLTSASYTMI

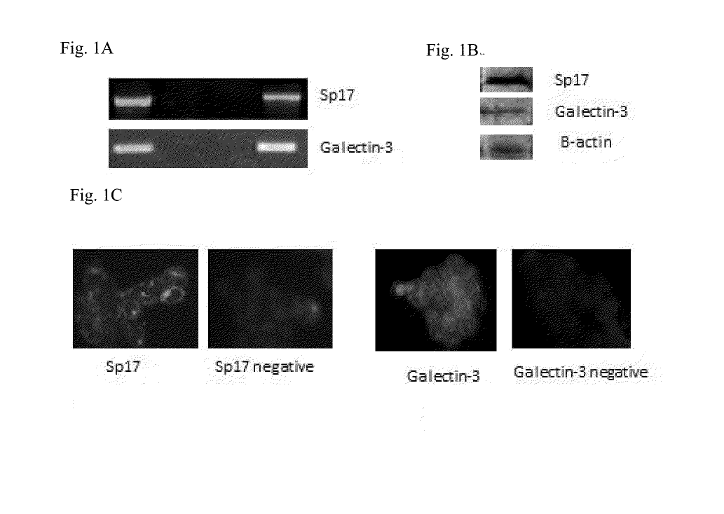

SP17 and Galectin-3 expression in 5TGM1 cell lines. RT-PCR (FIG. 1A) and Western blot (FIG. 1B) demonstrates that SP17 (a cancer testis antigen used as a biomarker in MM cells) and Galectin-3 are expressed in 5TGM1 cells at the mRNA and protein levels. Expression of both molecules by 5TGM1 cells was further confirmed by immunofluorescence analysis (FIG. 1C, left two panels) and co-localization (FIG. 1C, right two panels).

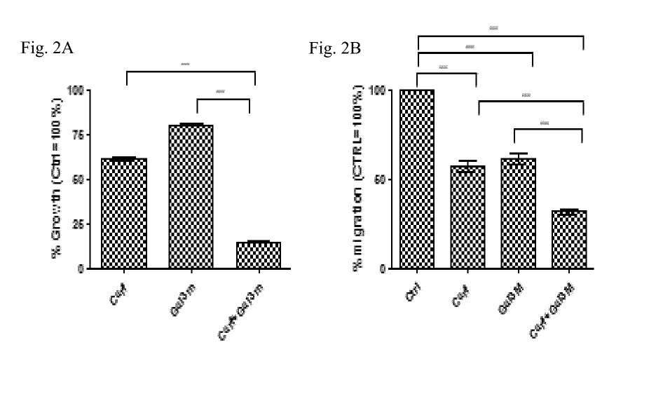

Gal3M and Carfilzomib inhibited 5TGM1 cells growth and migration. Results from viability and chemotaxis assays demonstrated that the double-treatment with carfilzomib Gal3M enhances the ability of each single compound to inhibit 5TGM1 cells growth and migration. The combined-treatment was associated with approximately 85% cell growth inhibition compared with the 20-40% growth inhibition caused by either treatment alone (FIGS. 2A and 2B). The ability of 5TGM1 cells to migrate in response to chemotactic stimuli was inhibited by approximately 65%, compared to 40% inhibition observed with either agent (FIG. 2B).

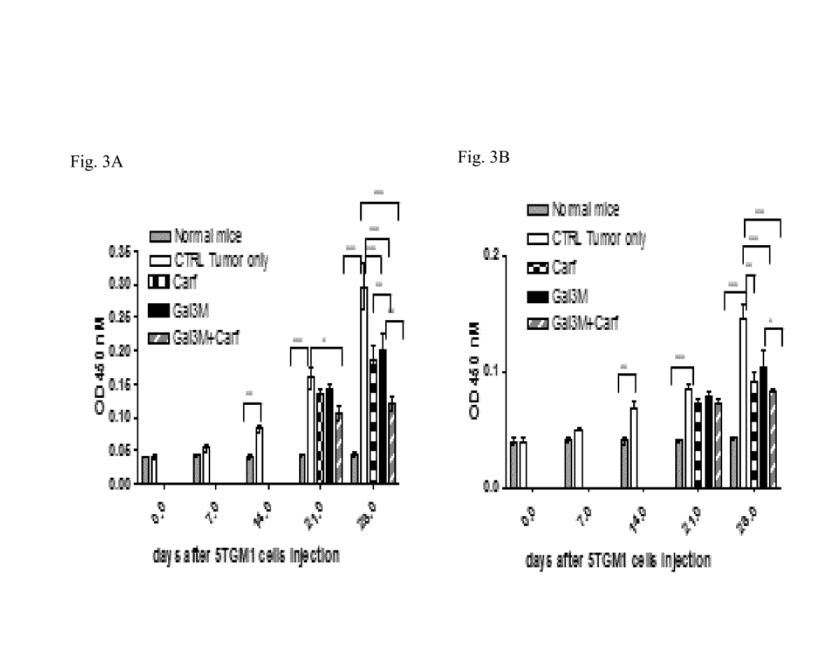

The effects of carfilzomib, Gal3M and the combination of these two compounds were evaluated in vivo, using the 5TGM1 syngeneic murine model of MM (FIGS. 3A and 3B). Mice were injected with 5TGM1 cells and by day 14 IgG2b levels increased, indicative of MM cell growth, engraftment and BM homing. Mice were treated with Carf (10 nM), Gal3M (10 ug/ml) or the combination of Carf+Gal3M (same doses) on day 14 and decreased in IgG2b levels were evident in all treatment groups at day 21 and 28, compared to untreated, MM hearing mice (14% and 36% decrease on Carf treated mice, 11% and 32% decrease on Gal3M treated mice, and 24% and 60% decrease for mice treated with the combination) (FIG. 3A). The combination of Carf+Gal3M was associated with additive inhibitory effects on IgG2b levels compared to either agent alone (FIG. 3A). Treatment with either Carf or Gal3M also resulted in decreased levels of SP17, compared to untreated mice (FIG. 3B). At day 28 SP17 serum levels were also lower in mice treated with the Carf+Gal3M combination compared to Gal3M, but not with Carf (43% decrease with Carf+Gal3M treatment, 37% decrease with Carf and 29% decrease with Gal3M).

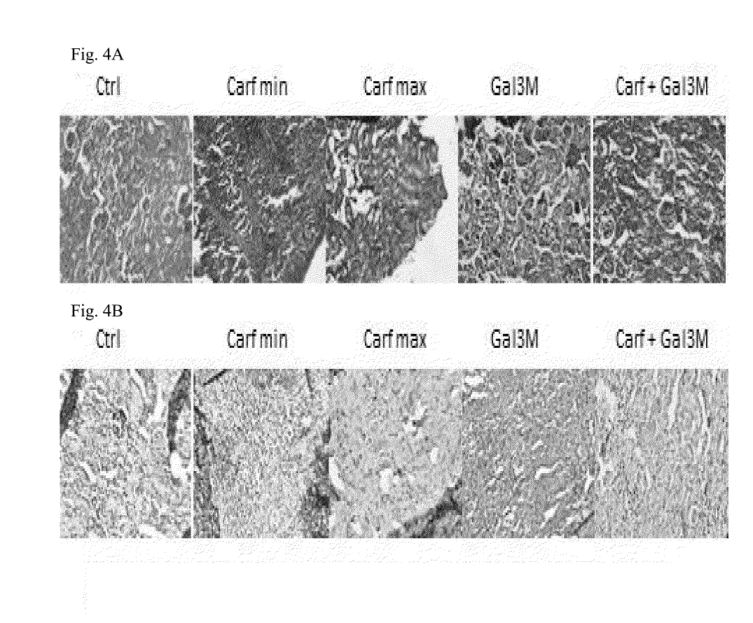

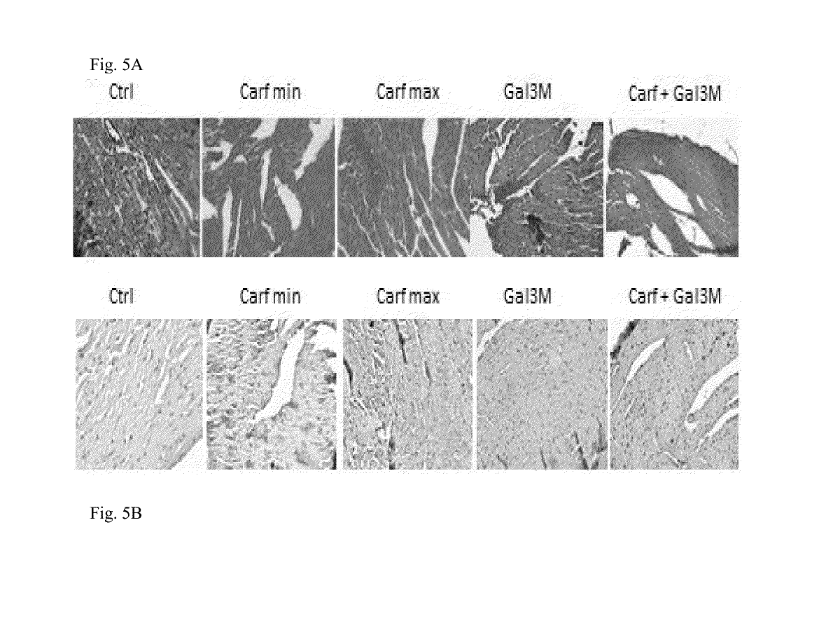

Gal3M protected the kidney and heart from Carfilzomib side-effects. To assess if Gal3M is able to inhibit the toxic effect of carfilzomib on the kidney and heart, we evaluated the morphology of these tissues with hematoxilin and eosin staining (FIG. 4A, kidney and FIG. 5A, heart) and assessed the presence of apoptotic cells with specific staining for activated caspase-3 (FIG. 4B, kidney and FIG. 5B, heart). Histological analysis indicated Gal3M is not associated with necrosis or increased apoptosis in the organs examined. Carfilzomib treatment, on the other hand, was associated with changes in cellular architecture and caspase-3 activation. Treatment with the carfilzomib-Gal3M combination resulted in a protective effect evidenced by a decreased number of cells staining for activated caspase-3.

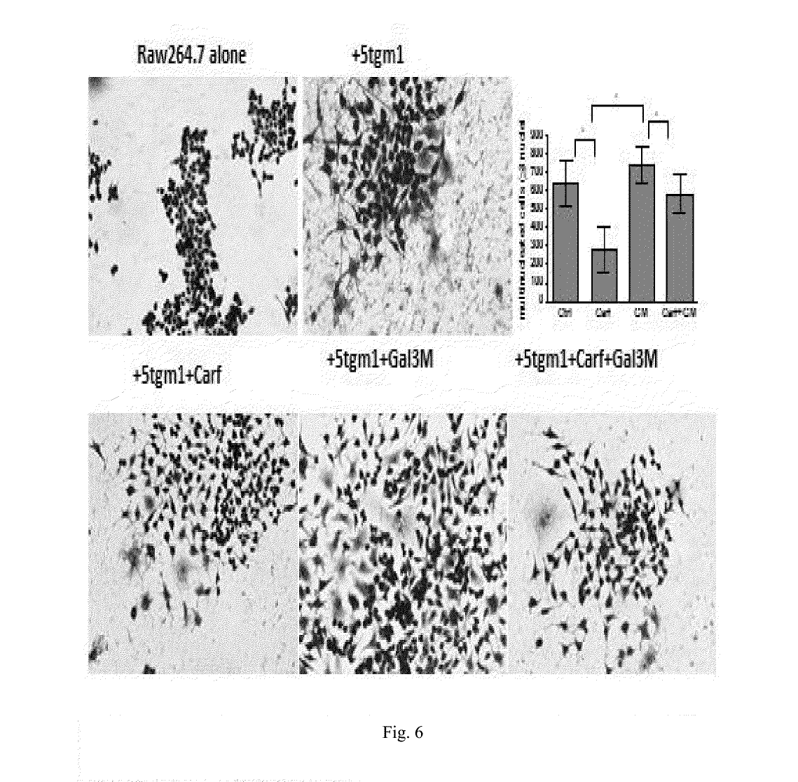

To investigate the effects of carfilzomib and Gal3M on MM-driven osteoclastogenesis, the 5TGM1 MM cell line was co-cultured with Raw264.7 cells with or without 5 μM carfilzomib, 10 μM Gal3M or the combination for 7 days. 5TGM1 cells induced the formation of TRAP⁺/multinucleated Raw264.7 cells (FIG. 6), which was inhibited by Carfilzomib (55% decrease) and enhanced by Gal3M (16% increase). The combination of carfilzomib-Gal3M treatment was able to decrease osteoclast formation compared with the control co-culture (9% decrease), indicating that carfilzomib is able to interfere with Gal3M pro-osteaoclastogenic effects.

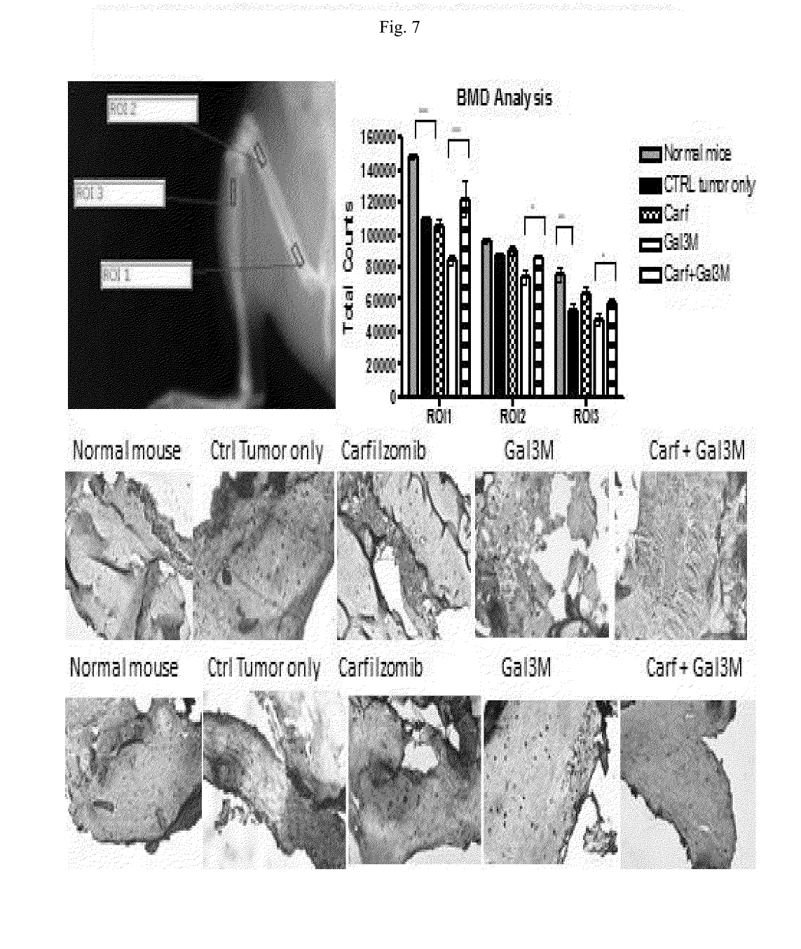

The combination of carfilzomib and Gal3M inhibits skeletal destruction in vivo. Bone mass density analyses were performed as previously reported (McManus M M, 2011) on x-ray images of control (no tumor) and 5TGM1 mice. The density of the proximal (ROI1) and the distal (ROI2) femur end and of the tibia (ROI3) were analyzed. The various panels of FIG. 7 show a BMD increase in mice treated with Carf alone (ROI1 28% increase; ROI2 19% increase; ROI3 12% increase) and a decrease in mice treated with Gal3M (ROI1 3% decrease: ROI2 4% decrease; ROI3 16% decrease). There was a significant increase in BMD after the treatment with the Carf/Gal3M combination (ROI1 10% increase; ROI2 2% increase; ROI3 8% increase) compared with tumor-bearing, untreated mice (FIG. 7). These data further confirm the ability of Carf to interfere with Gal3M pro-osteoclastogenic effect, protecting the bones from skeletal destruction.

FIG. 7 also shows the IHC analysis for osteocalcin (osteoblasts marker, B) and Cathepsin K (osteoclasts marker, C) on decalcified bones was performed to validate the BMD analysis. Indeed, mice treated with Carf showed an increase in osteocalcin-positive cells and a decrease in Cathepsin K-positive cells, confirming Carf-induced impairment of octeoclastogenesis and enhanced osteoblastic activity. On the other hand, Gal3M caused a decreased in osteocalcin-positivity and an increase in Cathepsin K-positive cells, supporting the pro-osteoclastogenic effect of this compound. The combined treatment shows that Carf is able to block this effect, leading to a decrease in Cathepsin K positive cells and to an increase in osteocalcin-positive cell population.

MM accounts for 10% of all hematologic malignancies (Rajkumar, 2012) and its incidence is constantly increasing due to the aging of the general population (Palumbo and Gay, 2009).

In the past decade, immunomodulatory drugs and proteasome inhibitors have extended the median survival to 5 years (Rajkumar, 2012). Unfortunately, and despite their effectiveness, both drug groups have serious side effects. Carfilzomib is a new irreversible proteasome inhibitor recently approved as single-agent treatment for relapsed or refectory MM. Despite its efficacy, carfilzomib has been associated with significant renal and cardiac side effects leading to discontinuation of therapy in approximately 15% of the treated patients (Redic, 2013). Indeed, cardiac failure has been reported in carfilzomib-treated patients, some of these events resulting in fatal outcome. Since approximately 60% of MM patients involved in carfilzomib clinical trials showed some degree of renal dysfunction at the time of enrollment, the need for careful patient selection and strategies that may result in decreased toxicity and organ protection when using this important drug is evident (Redic, 2013).

Galectin-3 has been shown to be involved in several biological processes in MM including cell growth, inhibition of apoptosis, cell adhesion and chemo-attraction. We have previously demonstrated the ability of Galectin-3C, a dominant negative inhibitor of Galectin-3, to inhibit MM growth in vitro and in viva (Mirandola, 2011). Galectin-3C alone and in combination with bortezomib resulted in more than 90% growth inhibition of MM growth in a xenograft mouse model. Importantly, Galectin-3 appears to have anti-osteoclastogenic effects (Li Y J, 2009) and is a mediator of both cardiac dysfunction (Ahmad, 2012) and renal fibrosis (Kolatsi-Joannou, 2011) in humans. Therefore, the interest in combining carfilzomib and Galectin-3 inhibitors in MM is justified based on several observations including their apparent additive anti-myeloma effects, carfilzomib's ability to inhibit osteoclastogenesis (Hurchla, 2013) resulting in neutralization the osteoclastogenic effects associated with Galectin-3 inhibition, as well as the potential cardio-renal protection that may be afforded by Galectin-3 inhibition.

This study used the syngeneic murine model of MM, 5TGM1, to evaluate the effects of carfilzomib combined with a new Galectin-3 inhibitor, named Galectin-3M (Gal3M). It was found that 5TGM1 cells, similar to the human MM cell lines used in the inventors' previous studies, express high levels of Galectin-3 and the cancer test is antigen SP17. It is shown herein that 5TGM1 cells were sensitive to the inhibitory effects of carfilzomib and Gal3M on their growth and migratory capacity. Surprisingly, treatment of 5TGM1 cells with the combination of carfilzomib and Gal3M resulted and additive inhibitory effects in these two cellular processes. These encouraging results prompted us to evaluate the in vivo effects of these treatments. 5TGM1 cells were injected in a syngeneic murine model and IgG2b and SP17 levels in mice sera analyzed. After 14 days a significant increase in IgG2b and SP17 levels was detect in the 5TGM1-injected mice engraftment and BM homing of MM cells. Mice were then treated with either carfilzomib, Gal3M, the combination or no treatment, and IgG2b and SP17 serum levels determined at 21 and 28 days post-injection on each group. These results, using this in vivo model, confirmed the inventors in vitro data. Surprisingly, the combined treatment with carfilzomib plus Gal3M was more effective than either agent alone in reducing IgG2b and SP17 levels, two markers of MM cell burden. These results support a powerful additive anti-myeloma effect when using carfilzomib and Gal3M.

In addition to assessing their anti-myeloma effects, the inventors evaluated the cardio-renal effects of carfilzomib and Gal3M in this murine MM model. Morphological analysis and IHC to detect caspase-3 activation (as an index of tissue damage and apoptosis) revealed that carfilzomib treatment was associated with morphological changes associated with kidney and cardiac tissue damage, as well as activation of the apoptotic pathway. Gal3M treatment was not associated with any obvious tissue damage and its addition to carfilzomib was able to protect both the kidneys and heart from carfilzomib-induced toxicity. These findings demonstrate the potential use of Gal3M, not only as an inhibitor of MM growth, but also as a protective agent against carfilzomib-mediated organ damage.

Bone destruction is one of the most common complications associated with MM, leading to hypercalcemia, generalized osteoporosis, skeletal pain and fractures. This interaction between the BM microenvironment and MM cells also results in the development a support of MM clones with enhanced growth and survival capacity, as well as drug resistance (Colombo et al., 2013). MM cells interact with the BM microenvironment causing increased osteoclastic differentiation and inhibition of osteoblast formation, favoring bone resorption and osteolysis. Since Galectin-3 has been shown to inhibit osteoclastogenesis (Li et al., 2009), the use of Galectin-3 inhibitors in MM may enhance and accelerate bone destruction in this disease. Therefore, whether the anti-osteoclastogenic and pro-osteoblastogenic proprieties of carfilzomib (Hurchla et al., 2013) could limit the skeletal effects of blocking Galectin-3 with Gal3M was also evaluated. The present invention showed skeletal effects of carfilzomib (increased osteoblastic activity) and Gal3M (increased osteoclastic activity) treatment both in vitro and in vivo. These results also demonstrated the use of carfilzomib in combination with Gal3M was associated with an osteoblastogenic, rather than osteoclastogenic mileu, resulting in bone formation, in vivo.

This study demonstrates the use of the Galectin-3 inhibitor, Gal3M, as an effective anti-myeloma agent, alone or in combination with carfilzomib. The use of Gal3M in combination with carfilzomib not only resulted in significant additive anti-myeloma activity, but was also associated with organ protection against carfilzomib-related toxicities. Importantly, carfilzomib treatment was able to counteract and overcome the osteoclastogenic effects of Galectin-3 blockade by Gal3M. In summary, these result shows that the use of Gal3M together with carfilzomib in a relevant MM, in vivo, resulted in additive anti-myeloma effects, cardio-As such, the combination of Gal3M and carfilzomib can be used for the treatment of patients with advanced MM.

Cells lines and treatments. The murine MM cell line 5TGM1 was a kind grit from Prof. Oyajobi Babatunde (Departments of Cellular & Structural Biology and Medicine, UTHSCSA, San Antonio, Tex., USA). The cells were maintained in 5% CO₂ atmosphere at 37° C. in complete IMDM medium supplemented with 10% V/V heat-inactivated FBS, 100 U/mL Potassium Penicillin and 100 μg/mL Streptomycin Sulfate. The macrophage/monocytes cell line Raw264.7 cell line was a kind gift of Professor Raffaella Chiaramonte (Dept. of Health Sciences, Università degli Studi di Milano, Italy). Cells were maintained in 5% CO₂ atmosphere in complete DMEM medium supplemented with 10% heat inactivated fetal bovine serum (PAA Laboratories, Inc). Carfilzomib was purchased from Onyx Pharmaceuticals, Inc., South San Francisco, Calif.

Construction of recombinant expression vector for Gal3M. Template cDNA was bought from genescopes and a primer pair for Galectin-3 mutagenesis was designed based to the nucleotide sequence of the Galectin-3 protein gene. Primers were designed in order to insert three different mutations at P113, L114 and Y118. Sequences were as follows: Forward; 5′GGATCCGCAGTGATTGTGCCTAATAACCT3′ (SEQ ID NO. 5), Reverse; 5′AAGCTTTATCATGGTATATGAAGCACTGGTG-3′ (SEQ ID NO. 6). The amplified fragment was inserted in the pQE30 vector.

Protein generation. Gal3M protein was generated through the use of pQE30/Gal3M plasmid transformed into M15 E. coli cells. IPTG (1 μM) was added as a promoter inducer once the cultures were grown and an O.D. of 0.6 was reached. Following growth of the E. coli cells, the protein was purified by Qiagen Ni-NTA Fast Start Kit.

Immunofluorescence. For the immunofluorescence analysis, cells were grown on sterile cover-slides coated with poly-L-lysine and fixed with 2% W/V paraformaldehyde in PBS for 5 minutes at room temperature (RT). Then, cells were blocked with 2% V/V FBS in PBS for 30 minutes and incubated with Galectin-3 or sp17 antibody (1:100, Kiromic LLC, Lubbock, Tex., USA) for 1 hour at RT. Slides were washed and incubated with a FITC-conjugated anti-mouse secondary antibody (1:1000, Becton Dickinson) for 1 hour at RT in the dark. After DAPI counter-staining, slides were mounted and pictures were taken at 60× magnification using an inverted Olympus X71 microscope.

Protein extraction and Western Blot. For protein extraction, 5TGM1 cells were lysated in cell lysis buffer (prepared mixing 2 ml of M-PER protein extraction buffer (Pierce/Thermo, Rockford, Ill., USA) with 40 ml of Protease Inhibitor Cocktail (Sigma-Aldrich). The cell suspensions were transferred to a new tube and incubated at room temperature (RT) for 10 minutes. Extracts were clarified by centrifuging at 12,000 rpm for 15 minutes, diluted to a final concentration of 2.5 mg/ml protein using M-PER, LDS buffer (Invitrogen, Carlsbad, Calif., USA), and the reducing agent, 2-mercaptoethanol, and then heated at 70° C. for 10 minutes. Proteins were resolved on 4-12% Bis-Tris (Bis(2-hydroxyethyl)-amino-tris(hydroxymethyl)-methane) polyacrylamide gel (Invitrogen) and then electrotransferred onto 0.2 μm nitrocellulose membrane. After rinsing in PBS-Tween, the blot was stored in protein-free blocking buffer (Pierce/Thermo) at 4° C. overnight. Then, the blot was incubated with anti-SP17 antibody (Kiromic LLC, Lubbock, Tex., USA) or anti-galectin-3 antibody (Kiromic LLC, Lubbock, Tex., USA.) diluted 1:200 in blocking buffer at RT for 30 minutes. After washing 5 times with PBS-Tween, the blot was incubated with biotin-conjugated anti-rat IgG diluted 1:2000 in blocking buffer at RT for 1 hour. After 5 washings, the blot was incubated with horseradish peroxidase-conjugated streptavidin diluted 1:1000 in blocking buffer at RT for 20 minutes, and then developed with 3,39,5,59-tetramethylbenzidine (TMB) with membrane enhancer (KPL; Mandel Scientific, Guelph, Canada). Images were captured with a digital camera and digitally enhanced (Adobe PhotoShop or ImageJ).

Cell viability assay. 5TGM1 cells were treated for 48 hours with carfilzomib, Gal3M or the combination of these two agents, then cell viability was assessed by the ViaLight Plus Cell Proliferation and Cytotoxicity BioAssay Kit (Lonza) according to the directions of the manufacturer.

Chemotaxis assay. 5TGM1 cells (4×105/well) were plated in 100 μL serum-free medium in the top chambers of 24-well Transwell™ inserts (Corning Costar, N.Y., USA) with 8-μm. Complete cell culture medium (600 μL) containing 10% FBS was added to the bottom chamber. Cells were treated for 4 hours with Gal3M (10 μg/ml), carfilzomib (10 nM) or carfilzomib plus Gal3M (same doses), and 5TGM1 cells in the lower chamber were fixed and counted.

Osteoclast differentiation from RAW264.7 cells. For co-culture experiments Raw264.7 and 5TGM1 cells were seeded on a 6-well plate at a density of 1×104 cells/well (about 8×103 Raw264.7 and 2×103 U266) and cultured in presence/absence of carfilzomib, Gal3M or carfilzomib plus Gal3M. After 5-7 days cells were fixed on the culture plates with citrate-acetone solution and stained for TRAP (Sigma-Aldrich). Osteoclasts were identified and enumerated under light microscopy by the presence of ≧3 nuclei by using an Olympus microscope.

Testing Gal3M and carfilzomib in 5TGM1 syngeneic mice model. 5TGM1 cells were washed once with PBS (Sigma-Aldrich) and counted. The suspension containing 107 5TGM1 cells was injected I.V. into 20 C57BL/KaLwRij mice, 5 non-injected mice were used as negative controls. Tumor engraftment was assessed by testing IgG2b in the sera once a week. After 14 days the injected mice were randomly divided into four groups (five mice/group). Control group #1 was injected with PBS-only, group #2 received a dose of Gal3M (100 μg/mouse), group #3 received a dose of carfilzomib (112 μg/mouse) and group #4 received a dose of carfilzomib (112 μg/mouse) plus Gal3M (100 ug/mouse). Treatment effect on tumor growth was assessed by testing IgG2b and SP17 levels in the sera. Anesthetized animals were euthanized after 28 days and a postmortem examination was conducted on the whole animals and dissected organs.

ELISA for Sp17 and IgG2b. The levels of SP17 and IgG2b in mice serum were measured by direct ELISA as follows. Flat-bottom 96-well polycarbonate plates were coated with 100 μL/well sera diluted 1:25 in carbonate coating buffer (0.1 M Na2CO3, 0.1 M NaHCO3, pH 9.5) at 4° C. overnight. Then, after blocking with PBS supplemented with 1% W/V BSA for 1 hour at RT, plates were incubated with mouse anti-mouse Sp17 or IgG2b primary antibodies (1:500 in PBS, 100 μL/well, Santa Cruz Biotechnology) for 1 hour at RT. Then, plates were washed thrice with PBS with 0.025% V/V Tween-20 (200 μL/well) and incubated at RT with anti-mouse HRP-labeled secondary antibodies (1:10,000 in PBS; Abcam, Cambridge, Mass., USA) for 1 hour. The plates were washed thrice with PBS containing 0.025% V/V Tween-20 (200 μL/well), the TMB colorimetric substrate (Thermo Fisher Scientific, Waltham, Mass., USA) was added, and after 5 minutes the absorbance was measured (0.1 s) at 450 nm on a Victor 2 multimodal microplate reader (PerkinElmer, Billerica, Mass., USA). All samples were run in triplicates.

Bone mass density analysis. X-ray images were taken at the euthanasia day using IVIS Lumina Series III Pre-clinical In Vivo Imaging System (Perkin Elmer, USA) and bone mass density analysis (BMD) were performed as previously described (McManus M M, 2011).

Immunohistochemistry. Immunohistochemistry analysis of markers of osteoclasts (anti-Cathepsin K antibody, Abcam), osteoblasts (anti-osteocalcin antibody, Abcam) and apoptosis (anti-activated caspase-3 antibody, Cell Signaling Technologies) were carried out in in paraffin-embedded sections. Hearts and kidneys from treated and control mice were fixed in formalin, dehydrated in ethanol, embedded in paraffin, and then cut in 3.5 μm-thick sections. For bone sections, following formalin-fixation, decalcification with 10% EDTA (pH=8), and de-hydration in ethanol, femurs were included in paraffin, then cut in 5 μm-thick sections. After deparaffinization in xylene and re-hydration (100%-95%-70%-50% ethanol scale), slides were placed 10′ in distilled water, then incubated 20′ at 98° C. in Sodium Citrate Buffer (10 mM Sodium Citrate, 0.05% Tween 20, pH 6.0). After blocking with PBS+5% V/V FBS (2 hours at room temperature), the anti Cathepsin K, steocalcin and active Caspase3 antibodies were added (diluted 1:400 in PBS+1% BSA) and allowed to incubate overnight at 4° C. Sections were rinsed twice in PBS+0.05% Tween-20, then endogenous peroxidase was blocked with 1% H₂O₂ in PBS for 15′. After rinsing three times with PBS+0.05% Tween-20 for 2′, slides were incubated with the secondary antibody (1:1000, Goat anti-Rabbit-HRP, Abcam) for 1 hour at RT. Staining was evidenced after a 5 minute incubation with 1X DAB solution. (Cell Signaling Technologies) followed by a 10-minute-incubation in distilled water. Pictures of hematoxylin counter-stained sections were taken by using an Olympus microscope.

Hematoxylin/Eosin staining. For morphological analysis hematoxylin (Sigma Aldrich, USA) and eosin (Sigma Aldrich, USA) staining was performed on heart and kidney paraffin-embedded sections. Slides were mounted using Permount mounting media (Fisher Scientifics) and images were observed under sight microscope (Olympus).

Statistical analysis. Data are represented as mean ±95% confidence interval (C.I.) of at least three independent experiments. Two-tailed Student's t-test was used to compare the means of normally distributed values. Analysis of variance was performed by Two-way ANOVA and Bonferroni post-test.

The Galectin-3M of the present invention lacks the N-terminal domain and does not multimerize, plus several mutations in the CRD region to increase affinity at the cell-cell and cell-matrix interaction.

FIG. 8 is a flow chart for a study of murine lung cancer with Gal-3M and Carfilzomib.

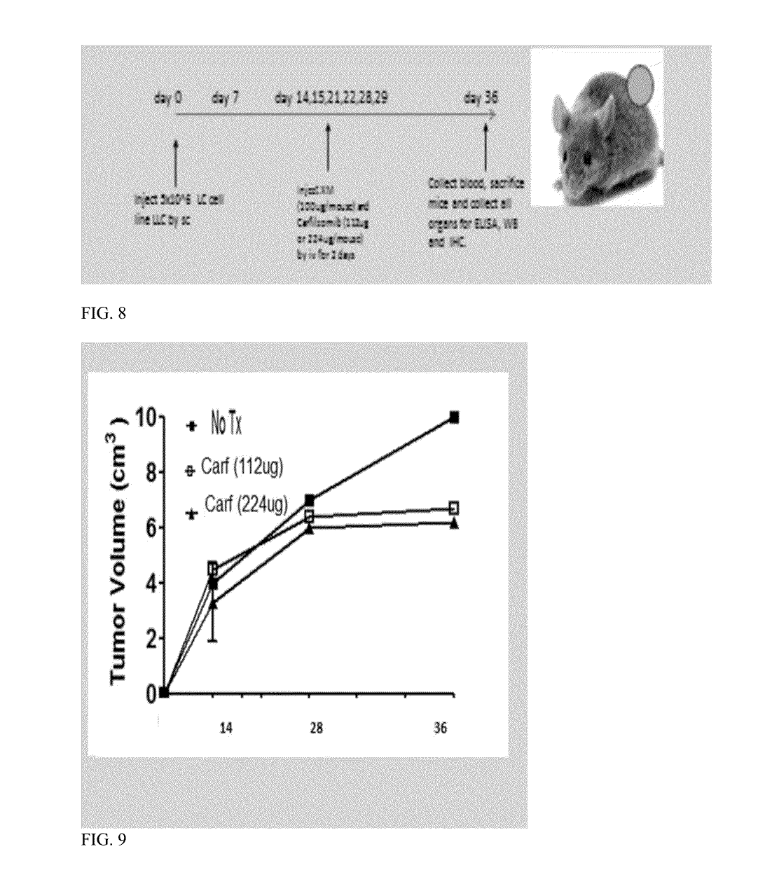

FIG. 9 is a graph that shows the change in tumor volume with no treatment and with 112 or 224 ug of Carfilzomib.

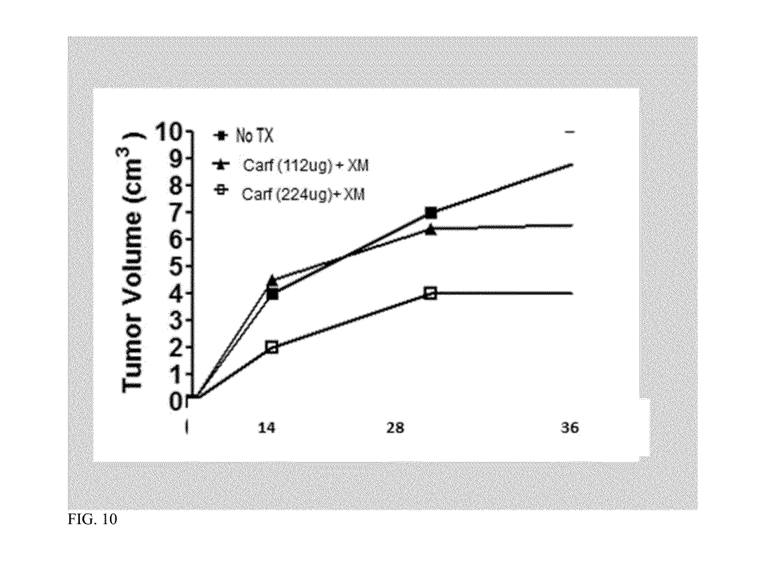

FIG. 10 is a graph that shows the change in tumor volume with no treatment and with 112 or 224 ug of Carfilzomib and XM.

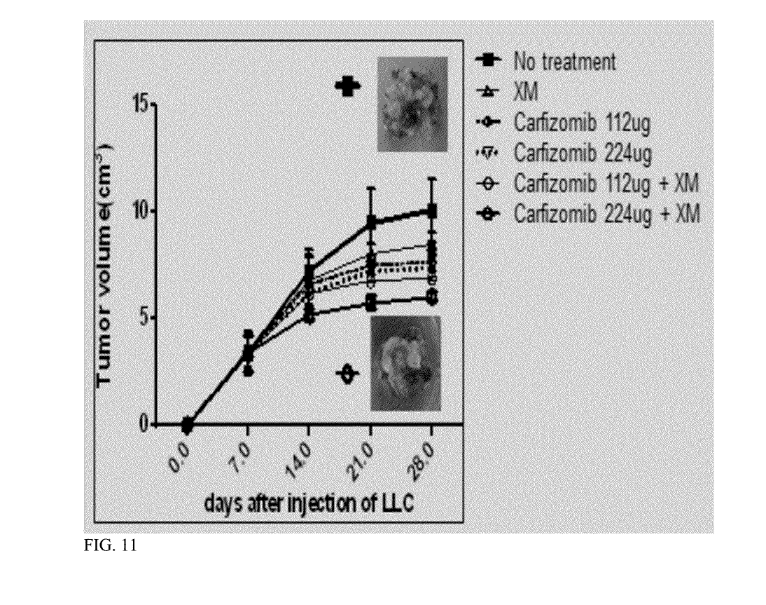

FIG. 11 is a graph that shows the treatment of a solid tumor with no treatment, XM alone, Carfilzomib alone (112 or 224 ug), and Carfilzomib 112 or 224 ug plus XM. Measurement of tumor size. 14 days after injection of LLC, the mice were treated with XM and Carfilzomib (112 ug and 224 ug) for two (2) consecutive days three (3) times weekly. The results show that XM, and Carfilzomib inhibited tumor growth.

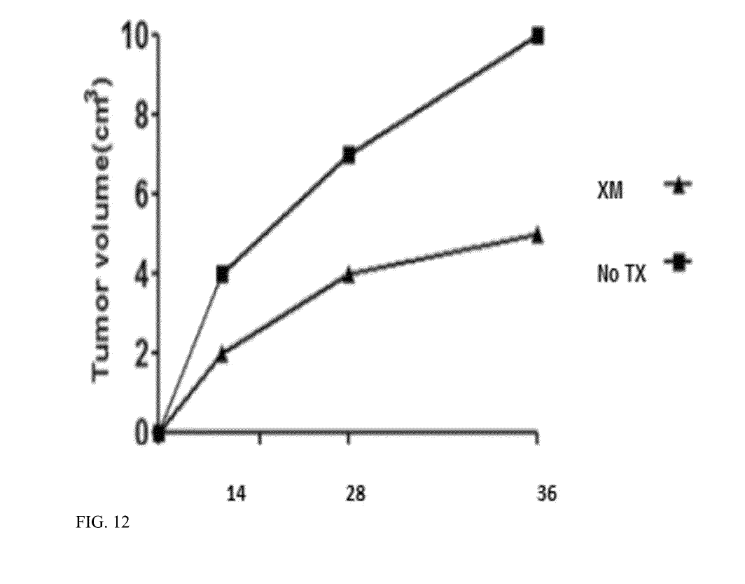

FIG. 12 is a graph that shows the change in tumor volume with no treatment and with treatment with XM.

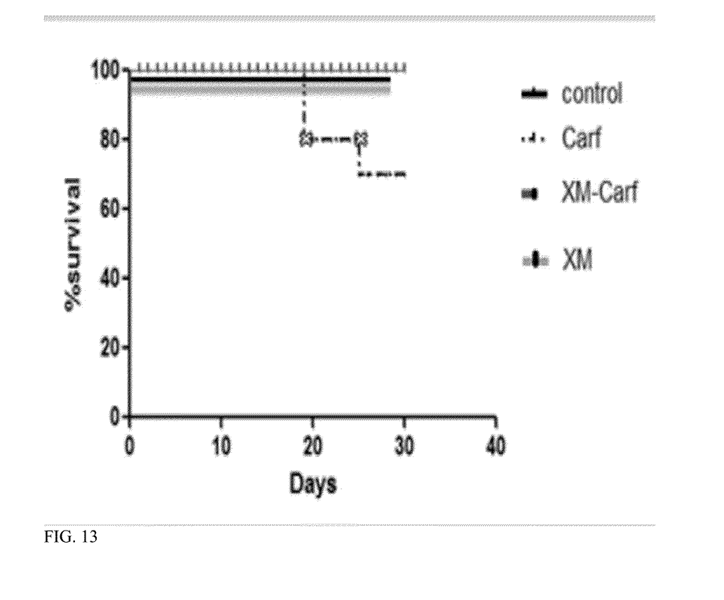

FIG. 13 is a graph that compares the toxicity of no treatment, XM alone, Carfilzomib alone, and Carfilzomib plus XM.

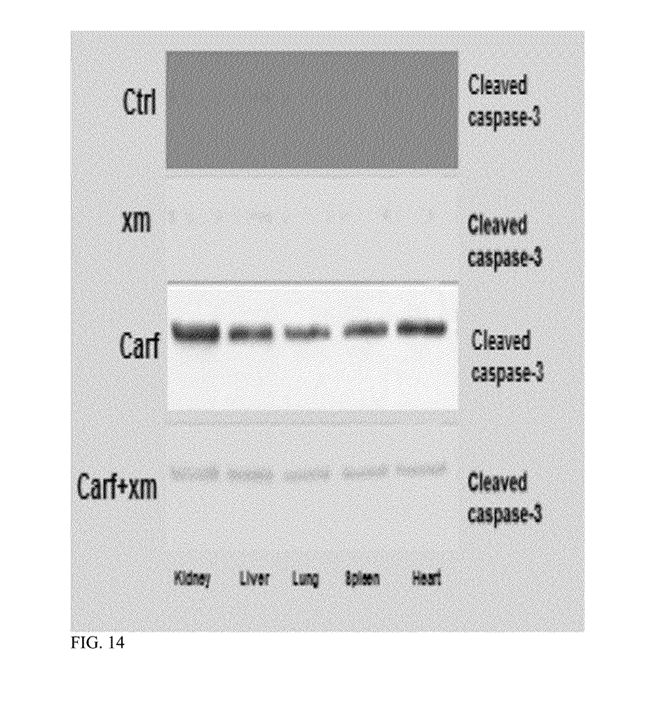

FIG. 14 is a Western Blot of the apoptopic marker caspacse-3 of cells in solid tumors with no treatment, XM alone, Carfilzomib alone, and Carfilzomib plus XM. After 36 days, the mice were sacrificed and organs collected for the Western Blot. Carfilzomib increased apoptosis in mouse organs such as kidney, liver, lung, spleen, and heart. XM reduced the apoptosis induced by Carfilzomib. There was almost no apoptosis in the control and XM groups.

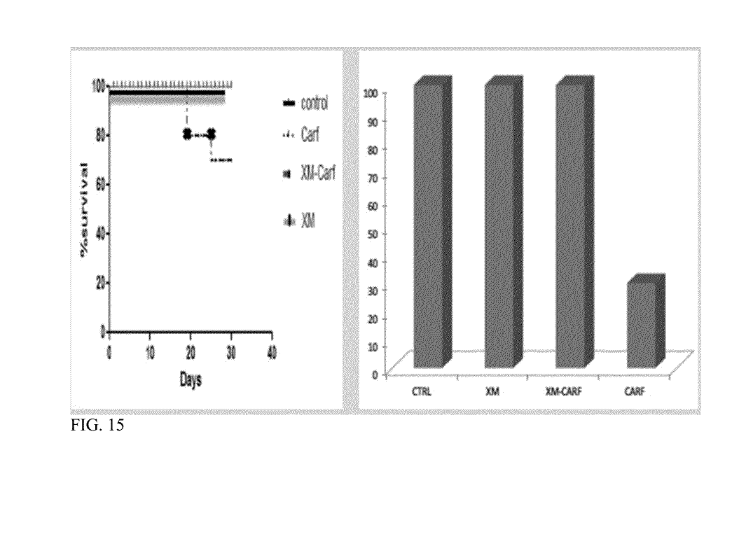

FIG. 15 shows two graphs, in the left panel the percentage survival of with no treatment, XM alone, Carfilzomib alone, and Carfilzomib plus XM (left panel), and on the right panel the toxicity of the no treatment, XM alone, Carfilzomib alone, and Carfilzomib plus XM.

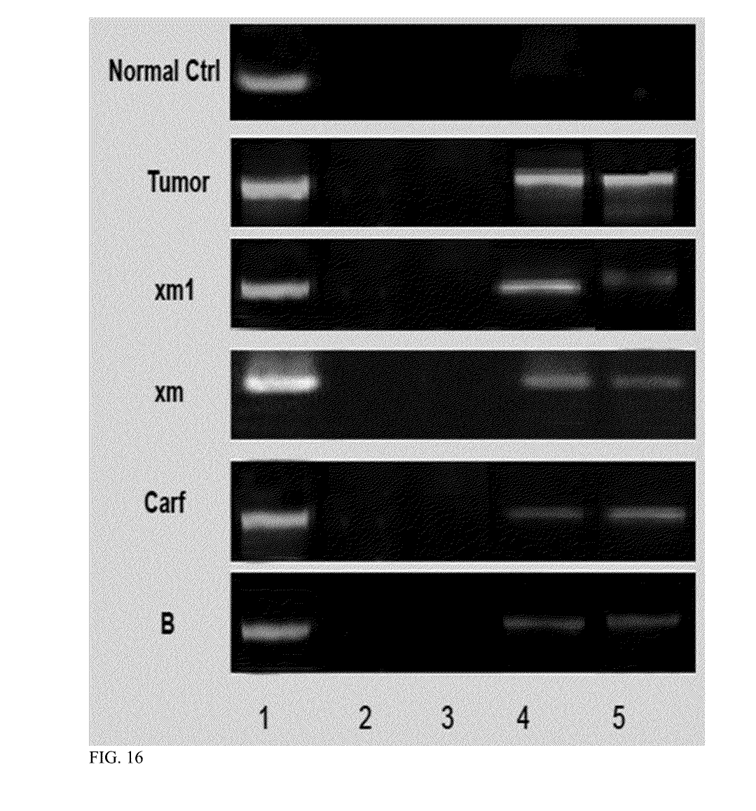

FIG. 16 is a reverse transcriptase-PCR for Sp17 in 5gtm mice, lane 1 5gtm1 cells, lane 2 no RT, lane 3 no template, lane 5 bone and lane 5 spleen.

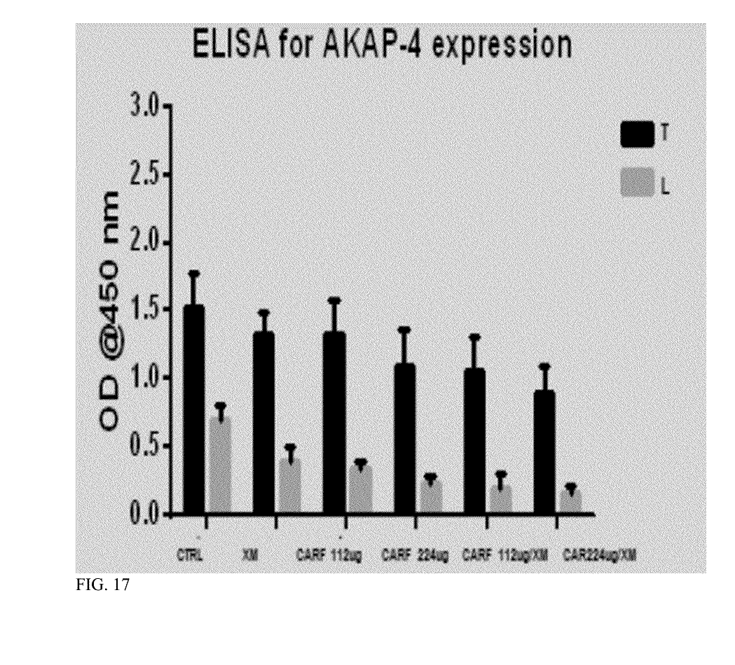

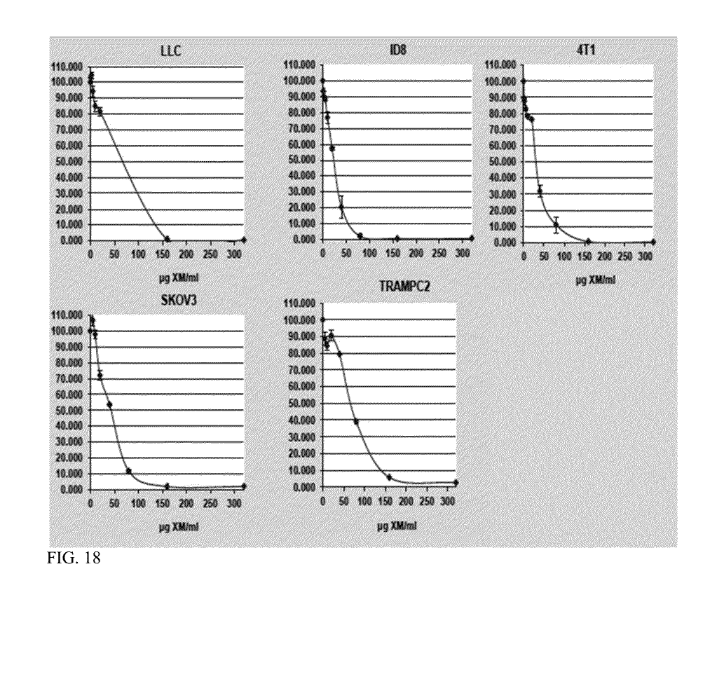

FIG. 17 is a graph that shows the results of an ELISA for AKAP-4 expression in tumor and lung tissue. The wells were coated with tumor and lung tissue lysates. The results show that the treatment with XM and carfilzomib have an effect on lung cancer (tumor).

FIG. 18 shows five graphs that show the expression of the listed cell lines as a function of the amount of XM provided.

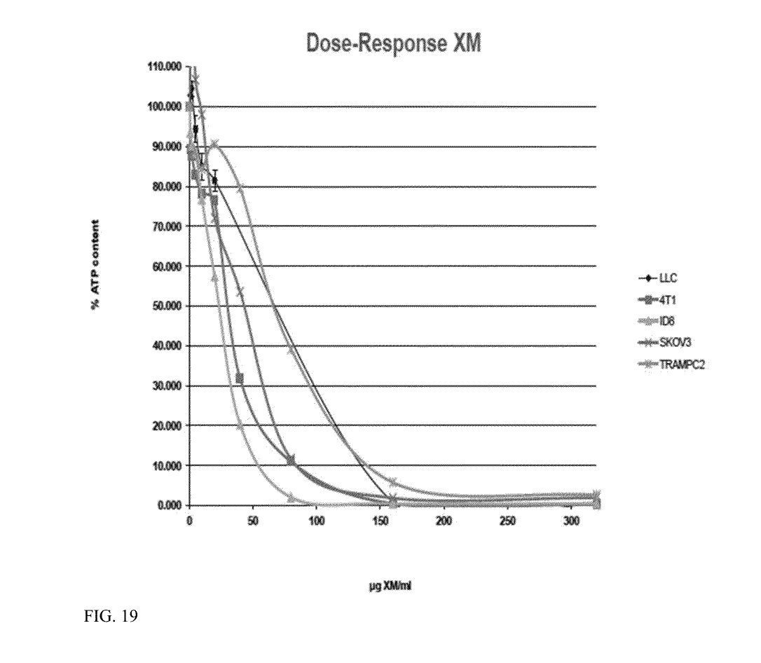

FIG. 19 shows a dose response curve of % ATP content for the various cell lines as a function of XM provided.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and parent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integers) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Ahmad, T., and G. M. Felker. 2012. Galectin-3 in heart failure: more answers or more questions? J Am Heart Assoc 1:e004374.

Colombo, M., L. Mirandola, N. Platonova, L. Apicella, A. Basile, A. J. Figueroa, E. Cobos, M Chiriva-Internati, and R. Chiaramonte, 2013. Notch-directed microenvironment reprogramming in myeloma: a single path to multiple outcomes. Leukemia 27:1009-1018.

Hurchla, M. A., A. Garcia-Gomez, M. C. Hornick, E. M. Ocio, A. Li, J. F. Blanco, L. Collins, C. J. Kirk, D. Piwnica-Worms, R. Vij, M. H. Tomasson, A. Pandiella, J. F. San Miguel, M. Garayoa, and K. N, Weilbaecher. 2013. The epoxyketone-based proteasome inhibitors carfilzomib and orally bioavailable oprozomib have anti-resorptive and bone-anabolic activity in addition to anti-myeloma effects. Leukemia 27:430-440.

Kolatsi-Joannou, M., K. L. Price, P. J. Winyard, and D. A. Long, 2011. Modified citrus pectin reduces galectin-3 expression and disease severity in experimental acute kidney injury. PLoS One 6:e18683.

Kyle, R. A., and S. V. Rajkumar, 2004. Multiple myeloma. N Engl J Med 351:1860-1873.

Landgren, O., and B. M. Weiss, 2009. Patterns of monoclonal gammopathy of undetermined significance and multiple myeloma in various ethnic/racial groups: support for genetic factors in pathogenesis. Leukemia 23:1691-1697.

Li, Y. J., A. Kukita, J. Teramachi, K. Nagata, Z. Wu, A. Akamine, and T. Kukita, 2009. A possible suppressive role of galectin-3 in unregulated osteoclastogenesis accompanying adjuvant-induced arthritis in rats. Lab Invest 89:36-37.

Mirandola, L., Y. Yu, K. Chui, M. R. Jenkins, E. Cobos, C. M. John, and M. Chiriva-Internati. 2011. Galectin-3C inhibits tumor growth and increases the anticancer activity of bortezomib in a murine model of human multiple myeloma. PLoS One 6:e21811.

Palumbo, A., and F. Gay, 2009. How to treat elderly patients with multiple myeloma: combination of therapy or sequencing. Hematology Am Soc Hematol Educ Program 566-577.

Rajkumar, S. V. 2012, Multiple myeloma: 2012 update on diagnosis, risk-stratification, and management Am J Hematol 87:78-88.

Redic, K. 2013. Carfilzomib: a novel agent for multiple myeloma. J Pharm Pharmacol 65:1095-1106, Saini, N., and A. Mahindra. 2013. Therapeutic strategies for the treatment of multiple myeloma, Discov Med 15:251-258.

What is claimed is:

1.-31. (canceled)

32. A protein comprising a Galectin-3 inhibitor having a sequence that is 95% identical to Galectin-3 with the following mutations P113, L114 and Y118.

33. The protein of claim 32, wherein the protein has 96%, 97%, 98%, 99%, or 100% sequence identity to Galectin-3 with the following mutations P113, L114 and Y118.

34. (canceled)

35. (canceled)

36. A host cell comprising a vector that expresses a Galectin-3 inhibitor having a sequence that is 95% identical to Galectin-3 with the following mutations P113, L114 and Y118.

37. The host cell of claim 36, wherein the host cell expresses the Galectin-3 inhibitor having a sequence that is 95% identical to Galectin-3 with the following mutations P113, L114 and Y118.

38. (canceled)
